(19) United States
(12) Patent Application Publication
(10) Pub. No.: US 2023/0260638 A1
(43) Pub. Date: Aug. 17, 2023

(54) FRAMEWORK FOR OPTIMIZING OUTCOMES FOR HEALTHCARE ENTITIES

(71) Applicant: Bloom Value Corporation, Norwalk, CT (US)

(72) Inventors: Arun Hampapur, Norwalk, CT (US); Sampoorna Hegde, Bengaluru (IN); Megha Kuchchal, Bengaluru (IN); Roona Shree, Bengaluru (IN); Narasimha Prasad Bhaskar, Bengaluru (IN); Sharathchandra Pankanti, Darien, CT (US)

(73) Assignee: Bloom Value Corporation, Norwalk, CT (US)

(21) Appl. No.: 18/169,232

(22) Filed: Feb. 15, 2023

(30) Foreign Application Priority Data
Feb. 16, 2022 (IN) 202241008272

(51) Int. Cl. G16H 40/20; G16H 50/70; G16H 50/30

(52) U.S. Cl. CPC G16H 40/20; G16H 50/70; G16H 50/30; G16H 10/20

(57) ABSTRACT

System and method for optimizing outcomes for healthcare entities, are described. In one aspect, the system includes an implementation of an optimization framework of machine learning engines, models, multiple sources of information or data. The machine learning engines may cooperatively collaborate with the models and execute operations on the data from the diverse data sources. In response to execution of the operations, the machine learning engines of the system may identify and/or determine multiple outcomes that may be optimized. The results of the execution of the operations by the machine learning engines of the system may provision analyzing different scenarios, analyzing recommendations, and take actionable steps based on the results of the analysis. Upon implementing the actionable steps, the outcomes for the healthcare entities may be optimized.

### CROSS REFERENCE TO RELATED APPLICATIONS/INCORPORATION BY REFERENCE

This application claims the priority benefit of an Indian Provisional Patent Application No. 202241008272, titled “FRAMEWORK FOR MANAGED RISK CONTRACT OPTIMIZATION”, filed on Feb. 16, 2022. The entirety of the above-mentioned patent application is hereby incorporated by reference and made a part of this specification.

### FIELD

Various embodiments of the disclosure relate to the technical field of data processing and data management, and more particularly to a system and method for optimizing outcomes for healthcare entities in this field. More specifically, the system and method may provide a framework for analyzing data, constraints, co-dependencies, morbidity conditions, high risk factors associated with the patients and provide insights to improve the quality of delivery of healthcare services.

### BACKGROUND

Conventional systems and practices in healthcare entities may include codependency on multiple factions and associated resources. However, when such healthcare entities scale up progressively with time, managing co-dependent factions may be cumbersome, complex, time consuming and operationally inefficient. Further, data associated with the factions may be related to co-dependent operations. Further, such conventional practices may include storing data or information related to patients, human resources, etc., in multiple dispersed data repositories, such as spreadsheets or local data storage systems. Such conventional practices may lead to creating working in silos and creating a lack of visibility in the data to optimally manage the healthcare entities. The complexity increases with an increase in a number of tasks in each faction of the healthcare entity.

The aforementioned practices that lead to complexities and silos in the technical field of data management may negatively impact the quality of service delivered by the healthcare entities. Further, due to lack of insights and systems to keep track of morbidity and high risk patients, the delivery capabilities and therefore the desired healthcare delivery objectives may be significantly impacted. Further, such a negative impact may yield lower return on investments (ROIs) due to misaligned information as the data is stored or managed through disparate systems, lack means for tracking of information due to the aforementioned storage techniques, and lack means to track metrics and measure the performance of the factions in the healthcare entities. Further negative impact may directly and adversely impact revenue or expenditures for healthcare entities, leading to a lower customer satisfaction levels, and an inadequate attention to the patients who may be at high risk. Consequently, it may be challenging to identify such co-dependencies and to measure the impact of changes in deliverables by the healthcare entities, thereby affecting an overall quality of service and making it difficult to undertake appropriate measures to improve outcomes.

The limitations and disadvantages of conventional and traditional approaches will become apparent to one of skill in the art, through comparison of described systems with some aspects of the present disclosure, as set forth in the remainder of the present application and with reference to the drawings.

### SUMMARY

A system and a method that implements embodiments of a framework for optimizing outcomes at healthcare entities is described. In an embodiment, the system and method provides an integrated framework of multiple, engines, models, circuitries, code executed by the circuitries, etc., to execute specific operations. Such operations may include analyzing data sourced from multiple data repositories, making computations and determinations based on the analysis and generating insights that are actionable. The system and method described in the subject specification may provision insights such that the value delivered by the healthcare entities is optimized.

The system and method described in the subject specification solves the technical problems associated with misaligned, siloed and incomplete healthcare data of the patients. The subject specification describes embodiments of system and methods that are in the technical field of data processing and data management. For example, the system and method described herein implement an execution of multiple engines, models, circuitries, code executed by the circuitries to optimize outcomes delivered by healthcare entities. For example, such optimizations may correspond to, for example, determining and timely intervening to treat high risk patients with prevalent diseases, determining utilization of budgets for healthcare related expenditures based on multidimensional data inputs, determining availability and utilization of human resources delivering healthcare services, etc.

The system and method described herein may further provide opportunities for optimizing use of time, money, materials, and human resources, such that healthcare entities are enabled to deliver the highest quality service while controlling costs and managing resources as efficiently as possible.

In the subject specification, the system and method described may be configured to receive a first data set from multiple data sources. For example, the first data set may be associated with multiple medical records associated with multiple patients. Further, the system and method may be configured to receive a second data set from the multiple data sources. For example, the second data set may be associated with multiple resources, an associated multiple attributes and an associated multiple constraints. Further, multiple engines and models in the system and operations executed by the method steps may analyze the received first data set in context of the received second data set. For example, the multiple engines or models may be implemented as multiple machine learning engines. Upon analysis, the system may be configured to generate one or more insights including one or more metrics associated with one or more tasks based on results of the analysis. For example, the one or more tasks are associated with one or more processes in a healthcare entity. Further, based on the generated one or more insights, the system may be configured to determine at least one task to be optimized from the one or more tasks. For example, the optimization may improve the one or more metrics associated with the at least one task.

In an embodiment, the machine learning engines may be trained based on training data related to the first data set and the second data set and re-trained continually based on an updated or modified first data set and an updated or modified second data set. Further, based on the analysis of the first data set in context of the second data set, the system may execute operations to generate a questionnaire to determine a treat plan for treating the patients. Further, based on the analysis of the first data set in context of the second data set, the system may execute operations to prioritize treating at least one patient from the multiple patients. For example, the prioritization of at least one patient may be based on multiple risk factors associated with the at least one patient. Further, the system may execute operations to modify at least one risk factor from the plurality of risk factors associated with the at least one patient based on the results of the analysis of the first data set in context of the second data set. Further, based on the results of the analysis of the first data set, the system may execute operations to generate a risk estimation associated with at least one patient from the multiple patients. Further, based on the results of the analysis of the first data set in context of the second data set, the system may execute operations to generate a schedule for treating the at least one patient.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a block diagram showing an environment 100 including an optimization framework 104 for optimizing outcomes in healthcare entities, according to an exemplary embodiment. FIG. 2A is a block diagram of a system 200A for optimizing outcomes for healthcare entities, according to an exemplary embodiment.

FIG. 2B is a block diagram showing an integrated system 200B to optimize outcomes for healthcare entities, according to an exemplary embodiment.

FIG. 3A is an illustration showing attributes associated with care service providers in a healthcare ecosystem, according to an exemplary embodiment.

FIG. 3B is an illustration showing attributes associated with a licensed provider service, according to an exemplary embodiment.

FIG. 3C is an illustration showing attributes associated with a care coordinator service, according to an exemplary embodiment.

FIG. 3D is an illustration showing attributes associated with a patient, according to an exemplary embodiment.

FIG. 4 is an illustration showing a representation of a healthcare service provided by a healthcare entity, according to an embodiment.

FIG. 5 is an illustration showing an organizational arrangement of the care coordinators, the licensed providers, the care services and the patients in a hospital ecosystem, according to an exemplary embodiment.

FIG. 6 is a flow diagram illustrating a process to schedule human resources at a healthcare entity, according to an exemplary embodiment.

FIG. 7 is a flow diagram illustrating a process for managing a patient treatment by a care coordinator service, according to an exemplary embodiment.

FIG. 8 is a flow diagram illustrating a process for treating a patient by a medical licensed provider, according to an exemplary embodiment.

FIG. 9 is a block diagram illustrating a system for generating a questionnaire for diagnosing a patient, according to an exemplary embodiment.

FIG. 10 is a block diagram showing a system for modifying a risk factor associated with a patient, according to an exemplary embodiment.

FIG. 11 is an illustration of an implementation of system at a healthcare facility in a specific demographic region, according to an exemplary embodiment.

FIG. 12 is a flow diagram illustrating a process for optimizing outcomes for healthcare entities, according to an exemplary embodiment.

FIG. 13 shows an exemplary hardware configuration of computer 1300 that may be used to implement components of the system (e.g., 100, 200A, 200B) for optimizing outcomes for healthcare entities, in accordance with exemplary embodiments.

### DETAILED DESCRIPTION

Embodiments of techniques related to a method, a system and a non-transitory computer readable device that implements an integrated system to optimize outcomes for healthcare entities, are described herein.

An accountable care organization (ACO) may include coexistence or cooperative working of primary health care providers, hospitals, and stakeholders of healthcare ecosystems to provide high quality healthcare services. Such high quality healthcare services may be provided to a group or a population of patients spanning over specific demographic regions. The objective of providing coordinated and collaborative high quality healthcare services is to enable the group of patients or a subset of the group of patients to receive the right kind and/or type of healthcare services at the right time, thereby optimizing the outcomes.

For example, such optimization of the outcomes may include, for example, prevention or elimination of duplication of healthcare services, minimizing or eliminating identification of errors and/or diagnostic oversights of the right population or a subset of right group of patients and improve the quality of healthcare services provided. Such a provision of coordinated and collaborative healthcare services may significantly reduce operational costs thereby improving profits and operating margins for various stakeholders in the healthcare entities, provide timely actionable insights for managing risks associated with lapses in early diagnosis or oversights in the healthcare, and managing tasks and actions such that the overall value in terms of quality and cost for providing healthcare services is optimized. Such optimizations of the outcomes may positively impact the provisioning in quality of healthcare, manage the cost related factors, reduce or eliminate risks associated with patient health and accountability of the ACOs.

In an embodiment, the subject specification describes a system or a framework that may also be referred to as an integrated optimization system or integrated optimization framework that may be deployed or implemented in a healthcare ecosystem. The system described may include multiple machine learning engines, machine learning models, artificial intelligence engines or models, optimization engines or models, estimation engines or models, computing engines or models that may be trained continually from multiple data sources. Further, the system may provide a simplified interface including user interfaces, dashboards, etc., that may provide insights that to aid making strategic decisions through engagement and actions that may optimize the value delivered by a healthcare entities.

In an embodiment, the integrated optimization system may include multiple engines, and models that may execute operations independently or in cooperation, to optimize the value delivered by the healthcare entities. The integrated optimization system may be configured to receive data from multiple data sources. The received data may be processed and analyzed by multiple engines, models, and the framework. Based on the analysis, the integrated optimization system may generate multidimensional insights associated with a measure of key performance indicators (KPIs). For example, the KPIs may be associated with multiple processes, services, tasks, etc., that may be delivered by the healthcare entity. Based on the generated insights, the integrated optimization system may be configured to determine multiple tasks and opportunities that may be optimized. Based on the determination, the stakeholders of the healthcare entities may act on the determined tasks and opportunities that may drive value or improvise the value/quality of service/process/tasks delivered by the healthcare entities.

In an embodiment, the integrated optimization system may provide and implement decision-based logics, multiple interfaces, engines and/or models, frameworks, one or more circuitries and/or code executable by the circuitries. The engines and/or models, frameworks, implemented by the integrated optimization system may execute operations either independently or in cooperation. An engine may correspond to a special purpose program or an executable code that performs or executes one or more core functions or operations. The engine may be continually trained by multiple data sources in real time or based on historical information or training data. The engine may be implemented as artificial intelligence engines or models or machine learning engines or models.

In an embodiment, modelling may correspond to a mechanism or a process that includes creating or improvising a functional or operational aspect of a system or one or more features of the system by referencing an existing or known knowledge base. The outcome of the modeling process may simplify the functional or operational aspect of the integrated optimization system or one or more features of the integrated optimization system that may be easily understood, quantified, and visualized. The mechanism for modeling may be automated through a continual process of training the model with data from multiple sources or data sources. The engines and/or the models may implement an execution of the one or more core functions or operations based on configured one or more rules and/or one or more sequence of sequence of steps to produce specific outcomes. The engines and/or models may be configured to work either independently or in conjunction with one or more engines or one or more models.

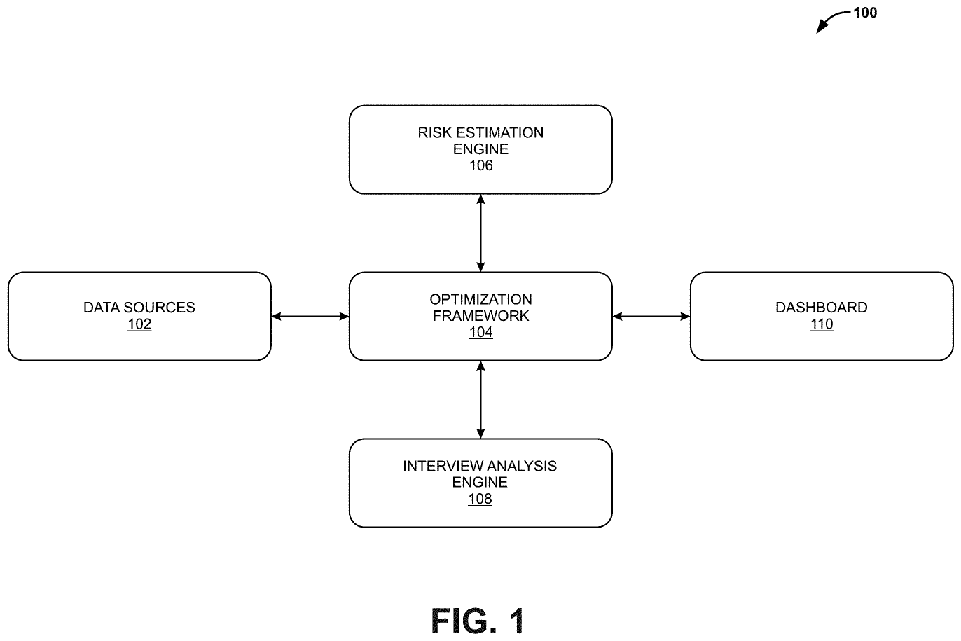

FIG. 1 is a block diagram showing an environment 100 including an optimization framework 104 for optimizing outcomes in healthcare entities, according to an exemplary embodiment. With reference to FIG. 1, there is shown a block diagram illustrating an environment 100 that includes an arrangement of communicatively coupled data sources 102, an optimization framework 104, a risk estimation engine 106, an interview analysis engine 108 and a dashboard 110. In an embodiment, the data sources 102 may store data or information from multiple repositories. For example, such repositories may include external data repositories, such as healthcare data, world health organization, national health services, center for disease control, etc. The data stored in the internal data repositories or data sources and/or the external data repositories or data sources may interchangeably be referred to as a first data set or a second data set. In an embodiment, the internal data repositories may store data related to, for example, clinical data, electronic medical record (EMR), administrative data, claims data, etc. The internal data repositories may further information or data associated with healthcare entities, patients, care coordinators, medical licensed providers, pharmacy, and other services providers known in the art that are associated with the healthcare entities. Such a data set may also be referred to as the first data set or the second data set.

In an embodiment, the optimization framework 104 may implement multiple models and engines that may be integrated and execute operations, either independently or in cooperation with specific objectives or outcomes. Further, the engines, the models, etc., may execute operations (e.g., analyzing, determining, etc.) based on multidimensional data inputs. Such multidimensional data inputs from the data sources (e.g., external data sources, internal data sources, etc.) that may be contextual in nature (e.g., based on socio-economic factors, availability of healthcare facilities in a specific demographic region, etc.) and/or dependent on multiple factors. In an embodiment, the multiple engines, models, etc., may be trained on the data set (e.g., the first data set or the second data set) in context of each other. For example, the operations such as analysis, determination, computations, etc., executed by the multiple engines, the models, etc., may be based on, for example, the first data in context of the second data or the second data in context of the first data.

For example, the multiple models in the optimization framework 104 may be associated with costs for different tasks such as screening procedures, disease management, treatment plans or processes, etc. The multiple engines may include quality management analysis engines, machine learning engines, estimation engines, optimization engines, etc. The risk estimation engine 106 in cooperation with the machine learning engines in the optimization framework 104 may execute operations on the data (e.g., the first data in context of the second data) and generate risk estimations associated with the patients based on multidimensional data analysis and computations. The interview analysis engine 108 in cooperation with the machine learning engines of the optimization framework 104 may execute operations on the data (e.g., the first data in context of the second data) to perform analysis of the results, for example, responses based on questions administered to patients.

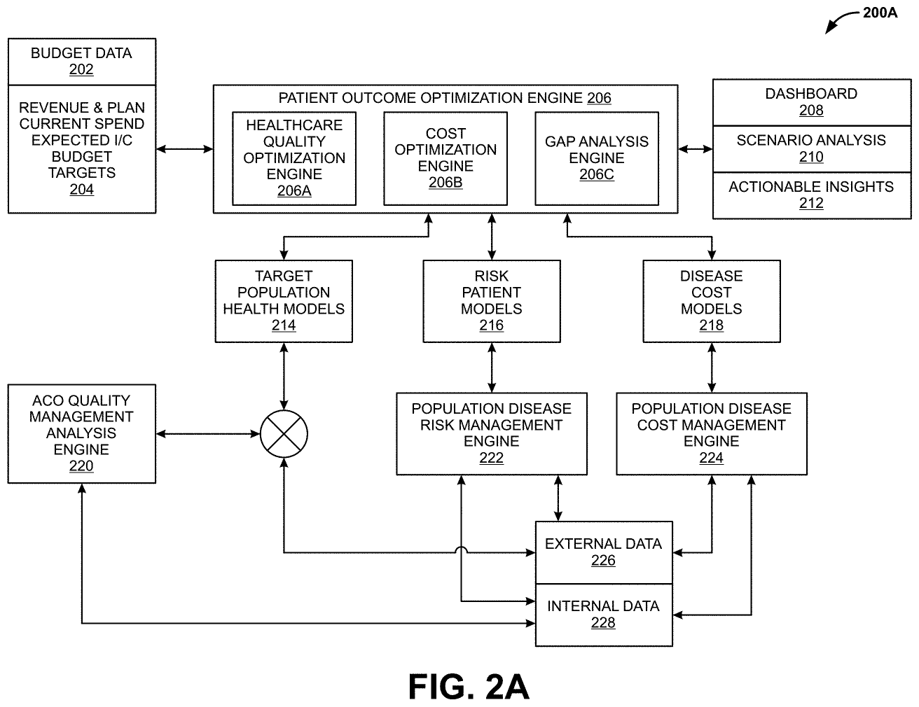

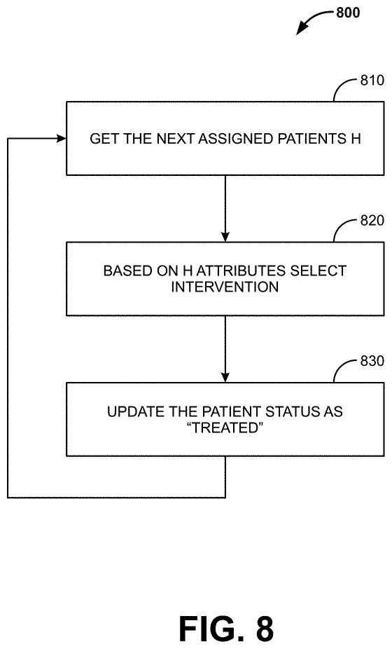

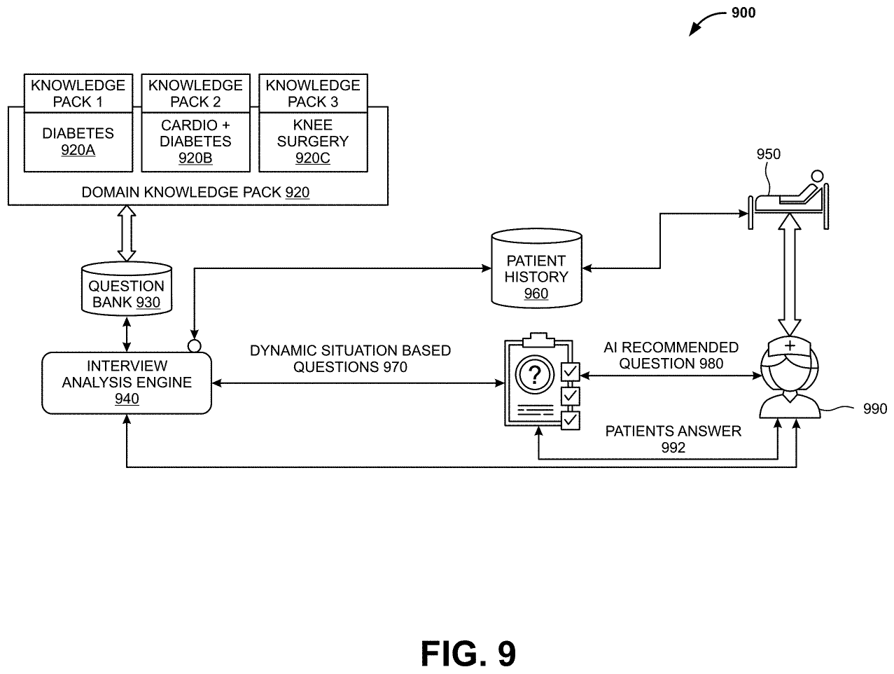

In operation, the multiple models and engines that may be described in the subject specification with reference to FIG. 1, FIG. 2A, FIG. 2B, FIG. 8, and FIG. 9 may further execute functions or operations, such as data normalization, analyzing and/or making determinations based on different circumstances and/or scenarios, analyzing and/or making determinations based on estimations, and analyzing and/or making determinations based on computations by using and applying context based information and data. For example, such data may include available resources (e.g., equipment, medical facilities, funds, assets, etc.), human resources (e.g., specialists, care coordinators, medical licensed providers, etc.), associated constraints (e.g., an unavailability, an availability, hours per week, budget, duration of availability, etc.), and associated attributes (e.g., skill level of the care coordinators, medical licensed providers, etc.).

In an embodiment, the multiple engines or models may execute operations on the data (e.g., the first data in context of the second data) to perform analysis and the results of the analysis may provide insights into one or more tasks that may be optimized. For example, the insights associated with the one or more tasks may generate actionable insights and recommendations for various stakeholders in the healthcare ecosystem. The actionable insights and recommendations may be displayed on user interfaces or dashboards on end user devices. The end users, for example, multiple stakeholders of the healthcare ecosystem, such as ACOS, doctors, decision makers, etc., may consume the results of the analysis and implement recommendations to enable optimizing the outcomes. In an embodiment, the term optimization in the subject specification may correspond to one or more of maximizing utility of human resources, and/or reducing risks associated with diagnosis and treatment of the patients, and/or minimizing constraints and dependencies without impacting the quality of healthcare delivery, and/or improvising the overall value in terms of cost, quality, and provision predictable treatment outcomes by the healthcare entities.

FIG. 2A is a block diagram of a system 200A for optimizing outcomes for healthcare entities, according to an exemplary embodiment. FIG. 2A is described in conjunction with FIG. 1. With reference to FIG. 2A, there is shown a block diagram of a system 200B for optimizing outcomes. In an embodiment, the system 200A may include multiple data sources (e.g., 202, 204, 226, and 228), an integrated optimization system including multiple engines and models (e.g., 206, 206A, 206B, 206C, 214, 2216, 218, 220, 222 and 224), and a dashboard 208. The data sources (e.g., 202, 204, 226, and 228) may source data or information from multiple repositories. For example, such repositories may include external data 226 repositories (containing external data from outside of the healthcare entities, such as healthcare data, world health organization, national health services, center for disease control, and including data such as healthcare data, world health organization, national health services, center for disease control, etc.) or internal data 228 repositories (containing internal data from inside of the healthcare entities and including data such as clinical data, electronic medical record (EMR), administrative data, claims data, patients, care coordinators, medical licensed providers, pharmacy, and other services providers). The external data 226 and the internal data 228 may be stored in multiple formats that may be normalized and processed by the engines and/or models in the system 200A. In an embodiment, the external data 226 may additionally store or include measures or metrics, for example, key performance indicators (KPIs) that measure a quality of tasks related to healthcare provisioning, a measure of information or data associated with treatment of prevalent diseases, such as cancer, a measure of cost associated with healthcare provisioning for diagnosis and/or treatment of the prevalent diseases, etc. For example, the metrics corresponding to the measure of quality of tasks related to healthcare provisioning may be defined and managed by national healthcare regulator, for example, healthcare effectiveness data and information set (HEDIS).

In an embodiment, the internal data 228 repositories may be private or restricted to a specific healthcare entity or a hospital that may be provisioning healthcare services in specific geographical areas. Such internal data 228 repositories may include information or data related to services rendered and associated costs, demographic coverage related information, patient population information, risk factor associated with the patients that is based on the patient's health conditions, lifestyle habits, socioeconomic factors, etc. Further, information or data may include influence of demographic factors such as Medicaid and disability status, gender, age, and whether a beneficiary or a patient is residing in a community or in an institution. For example, the patient or beneficiary may reside in a skilled nursing facility. Further, the information may include data corresponding to assessed risk, healthcare common procedure coding system (HCPCS), data associated with care coordinators, licensed providers, health care services, etc. Further internal data 228 repositories may also include information associated with a budget data 202, a current and planned current expenditures, an expected income, budgets targets 206, etc. The data stored in the external data 226 repositories and the internal data 228 repositories may store data that may be interchangeably referred to as the first data set or the second data set. The multiple engines and/or the models of the system 200A may execute operations on the data (e.g., the first data set in context of the second data set) to perform analysis and the results of the analysis may provide insights into one or more tasks that may be optimized. In an embodiment, the insights may include metrics associated with tasks corresponding to one or more processes that may be performed at the healthcare entities. The insights may be reviewed by stakeholders and one or more tasks may be determined to be optimized.

In an embodiment, the system 200A may include multiple models (e.g., 214, 216, and 218) and the machine learning engines (e.g., 206, 206A, 206B, 206C, 220, 222, and 224) that may execute operations or functions, either independently or in cooperation to optimize the outcomes. For example, the multiple models in the system 200A may include target population health models 214, risk patient models 216, disease cost models 218, etc. The above-described models (e.g., 214, 216, 218) may be continually updated and trained by modifications or updates in the data from the data sources (e.g., 204, 226, 228) in cooperation with the machine learning engines (e.g., 220, 222 and 224). For example, the target population health models 214 may be trained on modified or updated data, when an ACO quality management analysis engine 220 executes operations on the data (e.g., the first data set in context of the second data set). Further, the risk patient models 216 may be trained on modified or updated data, when the population disease risk management engine 222 may execute operations on the data (e.g., the first data set in context of the second data set). Further, the disease cost models 218 may be trained on modified or updated data, when the population disease cost management engine 224 may execute operations on the data (e.g., the first data set in context of the second data set).

In an embodiment, the disease cost models 218 may be trained based on data associated with costs. For example, such costs may be related to laboratory tests, radiology related screening procedures, disease management, treatment plans or processes, etc. The patient outcome optimization engine 206 may include a healthcare quality optimization engine 206A, a cost optimization engine 206B, and a gap analysis engine 206C which work cooperatively together, and which may work in cooperation with other engines and/or models in the system 200A. The patient outcome optimization engine 206 may execute operations on the data (e.g., the first data set in context of the second data set) to perform analysis and the results of the analysis may provision insights on the tasks or the outcomes that may be optimized. For example, the outcomes that may be optimized may enable implementing measures, modifying processes and associated tasks and improving associated metrics. For example, such optimizations may yield providing high quality healthcare services, determining redundant procedures that may add to cost of treatment, determining number of high risk patients that need time to time healthcare screening, benchmarking with national or geographic data for providing effective and strategic healthcare screening, providing an estimated cost of initial screening of high risk patients, providing an estimated cost, when potentially high risk patients are not screened on timely basis, providing estimated cost of treatment of specific types of diseases, providing or estimating patient outcomes such as survival rate, overall cost savings, etc.

In an embodiment, the engines of the system 200A may further include a patient outcome optimization engine 206 including a healthcare quality optimization engine 206A, a cost optimization engine 206B, and a gap analysis engine 206C. Further, engines may include an ACO quality management analysis engine 220, a population disease risk management engine 222, a population disease cost management engine 224, etc. The patient outcome optimization engine 206 by cooperatively working with the other engines in the system 200A may execute operations on the data (e.g., the first data set in context of the second data set) to perform analysis and the results of the analysis may provide insights into one or more tasks that may be optimized. For example, such optimizations may correspond to determining or identifying treatment plans with predictable outcomes. Further, the clinical outcomes for the patients may be associated with the quality of healthcare delivery. The patient outcome optimization engine 206 may execute operations on the data (e.g., the first data set in context of the second data set) to perform analysis and the results of the analysis may provide insights into one or more tasks that may be optimized. For example, the tasks to be optimized may provide insights associated with evidence-based resources. In an embodiment, such insights into the evidence-based resources may enable the care coordinators or medical licensed professionals to make faster and more adaptive decisions at the point of care.

In an embodiment, the healthcare quality optimization engine 206A in cooperation with other engines of the system 200A may execute operations on the data (e.g., the first data set in context of the second data set) to perform analysis and the results of the analysis may provide insights into one or more tasks that may be optimized. For example, the one or more tasks may be associated with optimizing the quality of healthcare delivery. Further optimizations may correspond providing insights for identifying or determining tasks or operations whose metrics or KPIs may be maximized or improved. The healthcare quality optimization engine 206A may execute operations on the data (e.g., the first data set in context of the second data set) to perform analysis and the results of the analysis may provide insights into one or more tasks that may be optimized. For example, insights may be related to tasks or processes to reduce or minimize disease prevention, for promoting health improvements, tasks or processes related to proactive chronic care management, etc. The cost optimization engine 206B may execute operations on the data (e.g., the first data set in context of the second data set) to perform analysis and the results of the analysis may provide insights into one or more tasks that may be optimized. Such optimization may be related to reducing costs for medical treatments and procedures. For example, optimizing costs may help in reducing the medical expenditures through timely intervention by the healthcare entities.

In an embodiment, the cost optimization engine 206B may execute operations on the data (e.g., the first data set in context of the second data set) to perform analysis and the results of the analysis may provide insights into one or more tasks that may be optimized. For example, the insights may be associated with one or more tasks, for example, for scaling healthcare support, making astute clinical decisions, making clinical workflow enhancements, etc., that may reduce costs for providing high quality healthcare. The gap analysis engine 206C may execute operations on the data (e.g., the first data set in context of the second data set) to perform analysis and the results of the analysis may provide insights into one or more tasks that may be optimized. For example, the insights may be associated with the one or more tasks that may help analyzing gaps in the healthcare services or processes. The gap analysis engine 206C may execute operations to provide insights related to examination and assessment of performance of the healthcare entities. For example, KPIs may be associated with identifying differences between current operations of services or processes and future/prospective operations or practices.

In an embodiment, the ACO quality management analysis engine 220 in cooperation with the other engines of the system 200A may execute operations on the data (e.g., the first data set in context of the second data set) to perform analysis and the results of the analysis may provide insights into one or more tasks that may be optimized. For example, such insights may be associated with the one or more tasks that may provision insights to optimize the quality of healthcare delivery. For example, the ACO quality management analysis engine 220 may execute operations for delivering high-quality care in a cost effective manner. Insights provided by the execution of the operations by ACO quality management analysis engine 220 may enable reducing per capita healthcare costs, improving health of populations, improving patient care, etc., may serve as KPIs associated with measuring the ACO quality management. The population disease risk management engine 222 may work in cooperation with other engines in the system 200A to execute operations on the data (e.g., the first data set in context of the second data set) to perform analysis and the results of the analysis may provide insights into one or more tasks that may be optimized. For example, the insights into the one or more tasks may reduce or optimize managing disease risk associated with the population of patients in specific demographic regions. For example, the operation of analysis may be executed based on data (e.g., the first data set or the second data set) including factors such as lifestyle, socio-economic factors, diagnosing and treating high risk diseases such as cardiovascular diseases, cancer, etc.

In an embodiment, the population disease cost management engine 224 may work in cooperation with other engines of the system 200A to execute operations on the data (e.g., the first data set in context of the second data set) to perform analysis and the results of the analysis may provide insights into one or more tasks that may be optimized. For example, the insights may be associated with the one or more tasks such as identifying and managing costs thereby preventing expensive medical expenditures. For example, the population disease cost management engine 224 may execute operations to provide insights into patient populations for a group of patients with chronic and high-risk conditions that may potentially lead to situations for increased expenditures. Such patients may be timely identified, followed up and treated, and the system may provide insights for tracking and managing their costs and care. In operation, the multiple models and engines shown of the system 200A may execute operations on the data (e.g., the first data set in context of the second data set) to perform analysis and the results of the analysis may provide insights into one or more tasks that may be optimized. For example, the results of analysis including insights including metrics associated with the tasks and/or processes may be rendered on the user interface or the dashboard 106. For example, the results of the analysis may include insights, recommendations on the outcomes, actionable steps, scenario analysis 210, and actionable insights 212 that may be associated with various scenarios. In an embodiment, the stakeholders of the healthcare entities may consume such insights and implement measures to optimize outcomes.

In an embodiment, the outcomes of the analysis provisioned as actionable insights 212 may be implemented by the respective stakeholders such that the overall value delivered by the healthcare entity is maximized. For example, the results of the analysis may correspond to various scenarios, for example, the scenario analysis 210 that may be optimized. Such scenarios may include predictable patient healthcare outcomes, quality of the healthcare provision of entire population or certain geographic areas, healthcare expenditures or cost of diagnosis and treatment, and expenditure margins for ACOS, etc. Further actionable insights 212 for the stakeholders and patients may include patient actions, healthcare provider actions, service provider actions, payor actions, etc. The actionable insights 212 may be able to optimize the operating margins for the ACOs. For example, the optimization may correspond to an increase in savings from an allocated budget for treatment of prevalent ailments. Further, the actionable insights 212 may improve quality of healthcare provision through cost optimized healthcare initiatives or programs. Further, the actionable insights 212 may be able to evaluate and manage provider performance through risk adjusted peer benchmarking. The ACOs may act or devise a strategy on the actionable insights and optimize the outcomes based implementation of processes driving the actionable insights 212.

In an embodiment, the system 200A may execute operations on the data (e.g., the first data set in context of the second data set) to perform analysis and the results of the analysis may provide insights into one or more tasks that may be optimized. For example, the results of the analysis may be related to foundational insights, healthcare insurance claims lifecycle, anomalies associated with healthcare insurance claims, frequency of claim denials, healthcare provider insights, billing provider insights, medical procedures and treatment procedure insights, action impact tracker, action dispatcher, etc. The results of the analysis may further include, for example, ACO specific insights, related to member utilization (e.g., resources or human resource utilization), provider utilization, medical insurance claims flow insights, insurance claims adjudication insights, patient health insights, etc. The results of the analysis may further include, for example, ACO business process and or tasks associated with the business process optimization insights, medical procedure cost optimization, provider utilization optimization, member utilization optimization, new patient risk analysis, patient health optimization, etc. The results of the analysis may further include, for example, ACO margin and quality optimization, related to financial predications, member health predictions, provider satisfaction metrics, etc.

In an embodiment, based on the data (e.g., the first data set or the second data set) from the external data 226 repositories and the data (e.g., the first data set or the second data set) from the internal data 228 repositories, the engines, for example, population disease risk management engine 222 may be configured to generate patient risk models 216. The patient risk models 216 may execute operations on the data (e.g., the first data set in context of the second data set) to perform analysis and the results of the analysis may provide insights into one or more tasks that may be optimized. For example, the insights associated with the one more tasks may enable providing an estimation of distinct types of risks associated with health of the patients based on different scenarios. For example, consider a scenario that may include providing an estimation of healthcare risk of the population of patients. Such healthcare risk estimation may be generated based on, for example, historical data related of the patients that may include data or information related to past health ailments, prevalent diseases, treatments, surgical procedures, etc., that the patients may have undergone. The machine learning engines in the system 200A may be trained with historical data and may implement multiple decision logics and rules to enable generating risk estimations associated with the population of patients. In an embodiment, the machine learning engines may be re-trained based on modified data from the data sources (e.g., the internal data 228 sources or repositories, the external data 226 source or repositories, the first data set, and the second data set).

For example, consider another scenario that may include generating risk estimations for the group or population of patients based on the lifestyle behavior of the patients. For example, such risk estimations may be computed based on risky behaviors such as negligence or oversight or overlooking health anomalies or associated health ailments, associated symptoms, etc. The above-described engines and the models may execute operations on the data (e.g., the first data set in context of the second data set, historical data, etc.) to perform analysis and the results of the analysis may provide insights into one or more tasks that may be optimized. In an embodiment, the historical data may further include information related to treatments or procedures follow ups that may have been overlooked or ignored by the patients. The historical data may also include data when the hospitals may have repeatedly followed up with the patients based on the treatment plans or procedures, and the patients may have ignored or have not been able to respond to such follow ups. Based on such data, the machine learning engines in the system 200A may determine risky behavior and indicate or flag such patients or population of patients.

For example, consider another scenario where the healthcare entities may actively intervene and engage with the patients or the population of patients to mitigate risks. The above-described engine and the models may execute operations on the data (e.g., the first data set in context of the second data set) to perform analysis and the results of the analysis may provide insights into one or more tasks that may be optimized. For example, the insights associated with the one or more tasks may provision insights into specific scenarios. Such a scenario may be a procedural treatment of an acute ailment or prevalent disease, such as cancer. The procedural treatment may include multiple steps in the treatment such as procedural scanning and treatment procedures like chemotherapy, radiotherapy, etc. In such scenarios, the healthcare entities may track and timely communicate with the patients, thereby leading to interventions for mitigating risks. Such active timely interventions may enable optimizing outcomes by implementing effective processes that may include proactive patient engagement leading to reduction in costs for laboratory procedures, radiology procedures, and pharmacy services.

For example, consider an implementation of the system 200A that may execute operations on the data (e.g., the first data set in context of the second data set) to perform analysis and the results of the analysis may provide insights into one or more tasks that may be optimized. For example, the insights may be associated with the one or more tasks for a scenario of screening procedures for a prevalent ailment, such as breast cancer. The screening procedures for breast cancer may be carried out for a specific demographic region. Further, consider that the demographic region may include a group or a population of 100 patients who may have demonstrated symptoms related to breast cancer. Of the 100 patients, consider that about 30 patients may have proactively participated in the screening procedures. Further, consider that the healthcare regulatory authority for that specific demographic region may have a minimum participation requirement for screening procedures of at least 50% of the patient population who may have or demonstrated symptoms related to the breast cancer.

In an embodiment, the multiple models (e.g., 214, 216, and 218) and the machine learning engines (e.g., 206, 206A, 206B, 206C, 220, 222, and 224) of the system 200A may execute operations to perform analysis, make computations based on constraints, availability of human resources, attributes, etc., make determinations, and provide results of the analysis. Further, the multiple models (e.g., 214, 216, and 218) and the machine learning engines (e.g., 206, 206A, 206B, 206C, 220, 222, and 224) of the system 200A may compare the results of analysis and determinations with the demographic participation statistics and generate actionable insights for the end users. The results of analysis by the multiple models (e.g., 214, 216, and 218) and the machine learning engines (e.g., 206, 206A, 206B, 206C, 220, 222, and 224) of the system 200A may further provide insights as to how many patients are at higher risk and may be contacted on priority to participate in the screening procedures.

Further, the actionable insights 212 may include cost of participation in the screening procedures by only a subset of remaining patients, for example, 25 patients from the remaining patient population of 70, such that the minimum participation requirements prescribed by the healthcare regulatory bodies are met. For example, a certain group or subset of population of patients may have comorbidities, such as diabetes, high blood pressure, etc. The actionable insights 212 may include such data or information associated with each patient. Further, the insights may include when to stop proactively encouraging the patients to participate in the screening procedures. Further insights may include the cost of not screening all potentially substantial risk patients.

Further actionable insights 212 may include the cost of treatment and survival rate of the patients. For example, the cost of treatment for a patient A who is diagnosed with breast cancer at stage 1 is about, for example, $20,000 USD and survival probability is about, for example, 80% upon treatment. Similarly, the cost of treatment for a patient C who is diagnosed with breast cancer at stage 4 is about, for example, $120,000 USD and survival probability is about, for example, 20% upon treatment. Further actionable insights 212 may include the budget to be allocated for screening procedures related to breast cancer, or other prevalent ailments, such as prostate cancer, high blood pressure, etc. In an embodiment, the results of the above analysis may be displayed on the dashboard 208.

In an embodiment, based on the actionable insights 212 displayed on the dashboard 208, the end user, for example, the ACO may devise a strategy to rank the patients, for example, 70 patients based on risk, prevalence data, comorbidities, etc. Further, the ACO may proactively communicate with, for example top 30 patients from the 70 patients and request for further information or data. For example, such data may be related to family history of major or prevalent health related ailments and other relevant information. Such additional data or information may be received by the system 200A and the machine learning engines, and the models may execute operations to perform computations to recalibrate or modify or update the results of analysis. Further, upon recalibration of the results, further determinations may be executed to provide new and/or further actionable insights. The new and/or further recalibrated results of the analysis and determinations may simplify the process of ranking and selecting a subset, for example, 20 patients from the remaining 70 patients. In an embodiment, based on the results of analysis, determinations, and the actionable insights, the ACO may be able to meet the minimum benchmark requirements by the healthcare regulatory authorities and also optimize the treatment of high risk patients and manage the budget allocated for screening substantial risk patients with prevalent health ailments.

FIG. 2B is a block diagram showing an integrated system 200B to optimize outcomes for healthcare entities, according to an exemplary embodiment. FIG. 2B is described in conjunction with FIG. 1 and FIG. 2A. In an embodiment, the integrated system 200B may include a communicatively coupled arrangement of multiple data sources (e.g., 203, 229), the integrated optimization framework (e.g., 104) and multiple dashboards or user interfaces (e.g., 208, 210, 212) that may be configured to render visualizations (e.g., 207). The multiple data sources (e.g., 203, 229) may include the internal data (e.g., 228) sources and external data (e.g., 226) sources, and also the data sources 202 and 204 as described above with reference to FIG. 2A, that may store data or information. The data from the above-described data sources (e.g., 226, 228) may interchangeably be referred to as the first data set or the second data set. For example, the data stored in 226 and 228 may include data related to patients (e.g., 230), electronic medical records (EMRs) related to the patients, demographic information including ease or difficulty of access to the healthcare facilities within a specific geographical area, historical data related to the patients including information related to disease risk levels of the patients, etc.

In an embodiment, the internal data (e.g., 228) sources and the external data (e.g., 226) sources may also store information associated with resources, for example, the socio-economic status of the group of patients (e.g., 230) or population of patients (e.g., 230) in the specific geographical areas, lifestyle factors, spending capacity of the population, assets owned by the population in the specific geographical area, etc. Further, the data stored may include information associated with an availability in terms number, diverse types, expertise and skill level of the licensed providers (e.g., 240), the care coordinators (e.g., 250), the care service (e.g., 260) providers, etc. Further, the data stored may also include information about availability the licensed providers (e.g., 240), the care coordinators (e.g., 250), the care service (e.g., 260) providers, etc. Further, the data stored may also include budget including revenue and plan current expenditures, expected income, budget targets, etc., that may be used as constraints when analyzing the information or data.

In an embodiment, the integrated optimization framework 104 may include multiple engines, models, multiple circuitries, logic, code, etc., that may implement an execution of specific operations or functions to achieve desired objectives. The engines, models, etc., may include multiple machine learning engines, analysis engines, risk management engines, cost management engines, optimization engines, etc. The above-described engines (e.g., 206, 206A, 206B, 206C, 214, 216, 218, 220, 222 and 224) may execute operations on the data (e.g., the first data set in context of the second data set) to perform analysis and the results of the analysis may provide insights into one or more tasks that may be optimized. The operational efficacies of the engines and the models (e.g., 206, 206A, 206B, 206C, 214, 216, 218, 220, 222 and 224) is as described with reference to the detailed description of FIG. 2A. In an embodiment, the outcomes may be displayed on dashboards or user interfaces that may be consumed by different stakeholders in the healthcare entity and further actions and steps may be taken to optimize the overall value delivered by the healthcare entities.

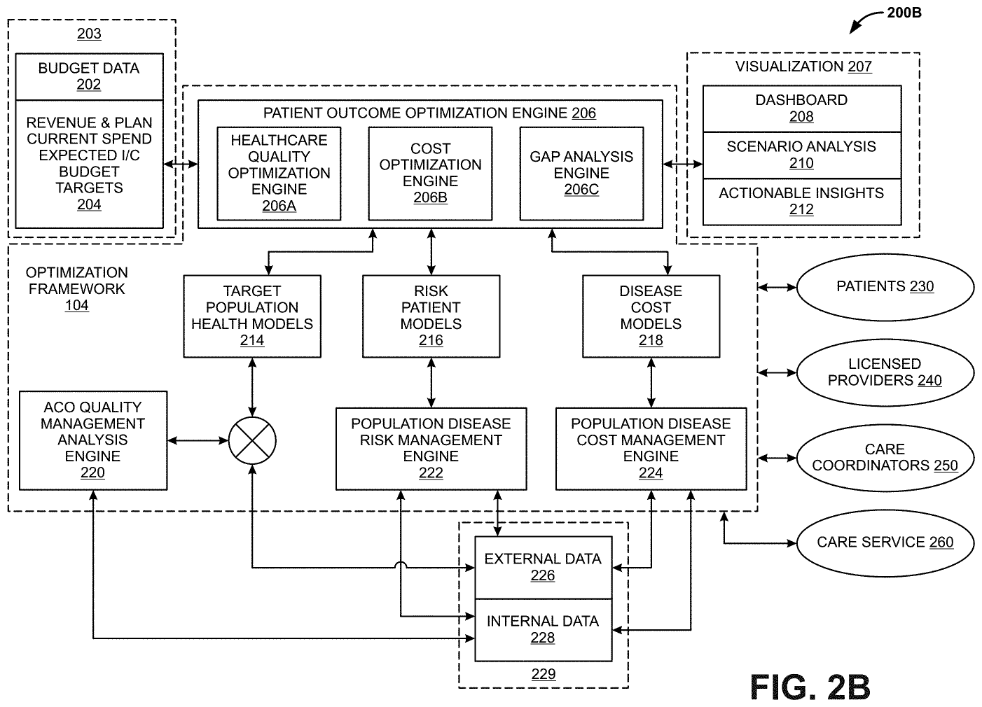

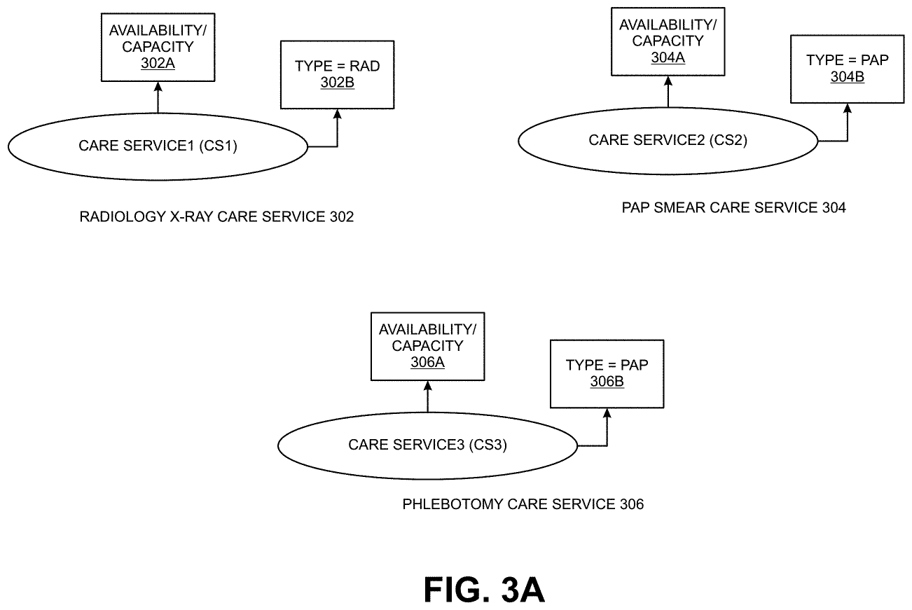

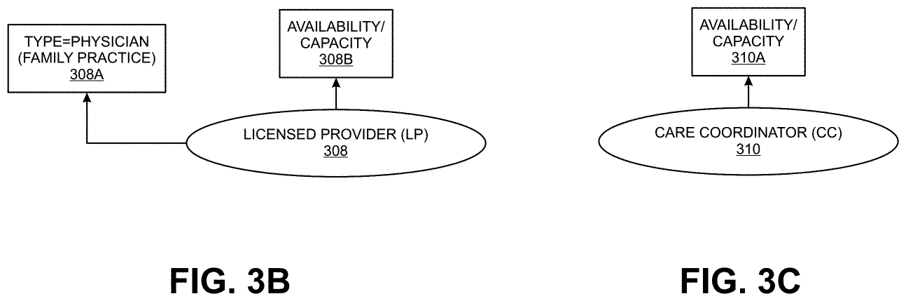

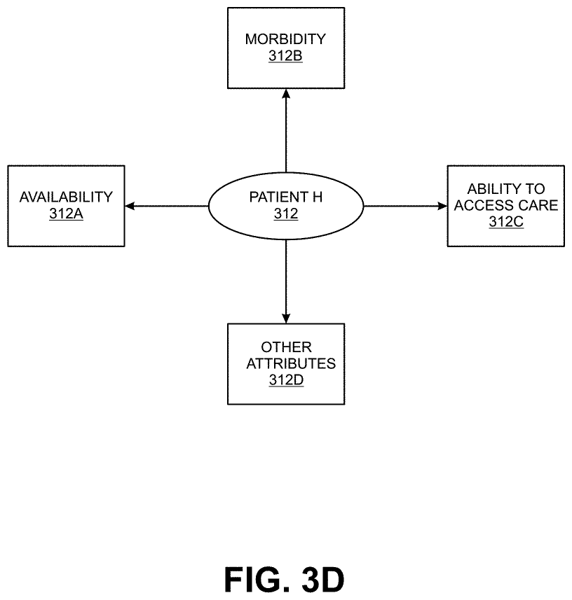

FIG. 3A is an illustration showing attributes associated with care service providers in a healthcare ecosystem, according to an exemplary embodiment. FIG. 3A is described in conjunction with FIG. 1, FIG. 2A and FIG. 2B. In an embodiment, a care service provider may provide assistance with obtaining laboratory test results for a patient. The care service providers may include a skilled staff that may be trained to handle specific equipment. For example, the care service may be of a type such as a radiology X-Ray care service 302, or pap smear care service 304, a phlebotomy care service 306. In an embodiment, the types of services provided by the care service providers may be narrowed down based on the specialization such as X-ray in the radiology care service 302. Each care service provider may operate and provide services as a single unit that may further include the corresponding staff for operating the equipment. Each care service provider may be associated with a corresponding attributes such as a type of service (e.g., 302B, 304B, 306B) and an availability/capacity (e.g., 302A, 304A and 306A).

FIG. 3B is an illustration showing attributes associated with a licensed provider service, according to an exemplary embodiment. FIG. 3B is described in conjunction with FIG. 1, FIG. 2A and FIG. 2B. In an embodiment, the licensed provider (LP) 308 service may also be referred to as a medical licensed provider service who may assist in clinically assessing a patient. The licensed provider 308 service may cooperatively work with the skilled staff who may be trained to operate diverse types of equipment. Further, the medical licensed provider service could be of type physician service 308. The types (e.g., 308A) of services could be further specialized such as family practice type of physician service. Each licensed provider service may operate and provide services as one single operational unit that may further include the corresponding staff and equipment needed to provide the service. Each provider service may be represented by its corresponding attributes such as a type of service (e.g., 308A) and an availability/capacity (e.g., 308B).

FIG. 3C is an illustration showing attributes associated with a care coordinator service, according to an exemplary embodiment. FIG. 3C is described in conjunction with FIG. 1, FIG. 2A and FIG. 2B. In an embodiment, a care coordinator (CC) 310 may help coordinate the patient's overall care. The care coordinator service may additionally include equipment and servicing staff. Each care coordinator service may operate and provide services as a single unit and may include additional staff and equipment needed to provide services as care coordinators. Each care coordinator service may be represented by its corresponding attributes such as an availability/capacity (e.g., 310A).

FIG. 3D is an illustration showing attributes associated with a patient, according to an exemplary embodiment. FIG. 3D is described in conjunction with FIG. 1, FIG. 2A, and FIG. 2B. For example, the attributes associated with the patient may include, for example, an availability (312A), morbidity 312B (e.g., diabetes, blood pressure, etc.), ability to access care services (e.g., 312C), and other attributes 312D, for example, types of sicknesses, associated electronic medical records (EMRs), etc.

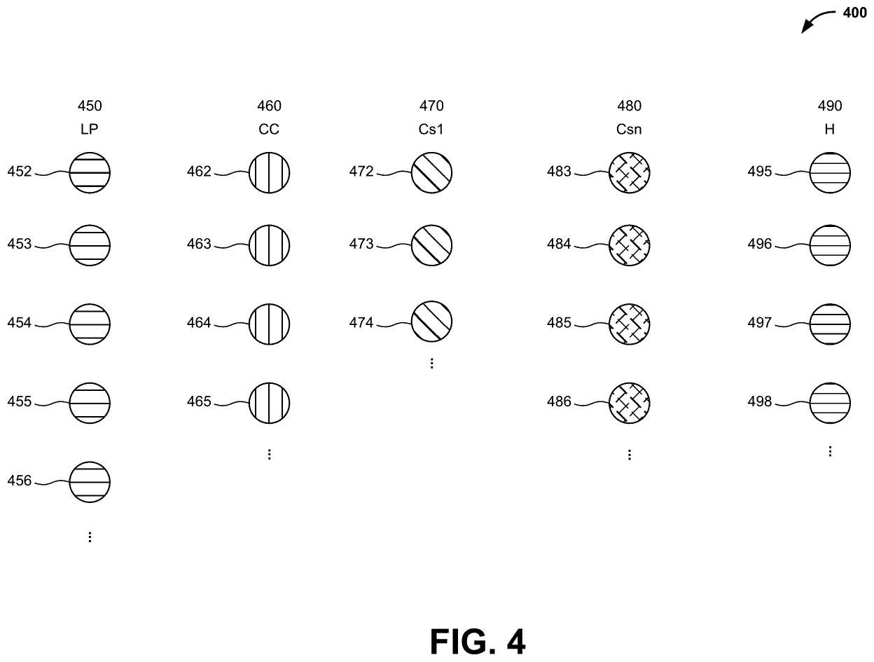

FIG. 4 is an illustration showing a representation of a healthcare service provided by a healthcare entity, according to an embodiment. FIG. 4 is described in conjunction with FIG. 1, FIG. 2A, and FIG. 3B. With reference to FIG. 4, there is shown a representation of healthcare/care service provided by the healthcare entity or the hospital. In an embodiment, the healthcare entity or the hospital may include one or more medical licensed providers (LP) 450 (e.g., 452, 453, 454, 455, 456) providing licensed providers service. Further, the healthcare entity may include care coordinators (CC) 460 (e.g., 462, 463, 464, 465) providing CC services. Further, the healthcare entity may include multiple care services (CS1) 470 (e.g., 472, 473, 474, CSn 480, 483, 484, 485, 486), and multiple patients (represented as ‘H’ 490, 495, 496, 497, 498). In an embodiment, each of the above described may be represented by columns of dots and corresponding reference numerals.

In an embodiment, when a new patient or service provider, for example, a new care coordinator or a new care service, a new license provider, etc., is onboarded, the internal data source associated with the hospital may be updated and a corresponding representation may be updated to include the inclusion. In an embodiment, upon such inclusion, the workflow for managing the patients in the hospital may be modified. For example, a patient registration process in the existing workflow may be modified to introduce associated patient record within the overall system. In an embodiment, when a new equipment is purchased or a new licensed provider is hired, such an event may instantiate a process of updating or modifying associated representations within the overall hospital ecosystem. In an embodiment, the licensed providers, the care coordinators, and the care services may be available to attend to the patients based on their work schedule. When the licensed providers, care coordinators, and care services are attending to patients and providing the assigned or respective services, the corresponding units of the period may be reflected as unavailable in the system.

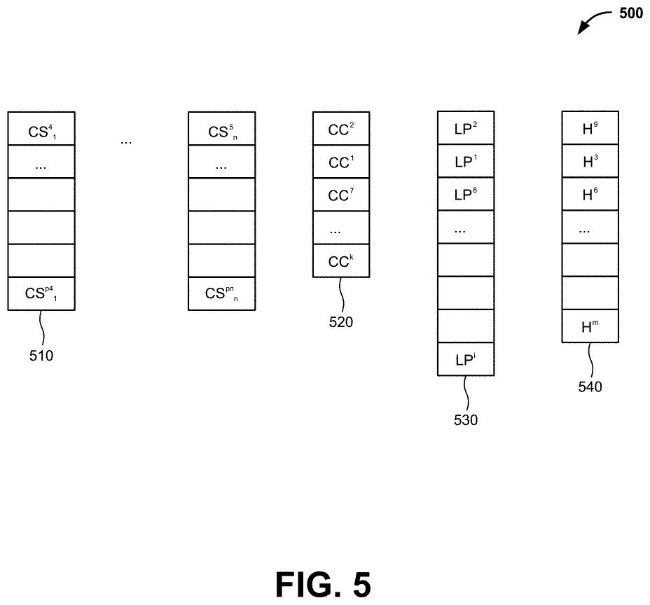

FIG. 5 is an illustration showing an organizational arrangement of the care coordinators, the licensed providers, the care services and the patients in a hospital ecosystem, according to an exemplary embodiment. FIG. 5 is described in conjunction with FIG. 1, FIG. 2A, FIG. 2B, FIGS. 3A-3D and FIG. 4. In an embodiment, each resource (e.g., the care coordinator, the licensed provider, the care service and the patient) may be represented and organized based on an availability in a next instance. For example, instances of each type of care service (e.g., CS⁴₁, CS⁵ₙ, CS^{pn}ₙ, etc.) may be organized separately 510. In an embodiment, an availability of the care coordinators in the next instance (CC) instances may be represented as shown 520 (e.g., CC², CC¹, CC⁷, CC^{K}, etc.), an availability of the medical licensed providers (LP) in the next instances may be shown as 530 (e.g., LP², LP¹, LP⁸, LPⁱ, etc.), and the availability of patients (H) for treatment in the next instances may be shown as 540 (e.g., H⁹, H³, H⁶, H^{m}, etc.). In an embodiment, the hospital ecosystem may provision tracking and searching the availability of each human resource (e.g., CC, CS, LP) at the next instance. Such organizational arrangements may be analogous and conceptualized as multidimensional queues. In an embodiment, FIG. 5 may merely illustrate the representation of the resources (e.g., care coordinators, medical licensed providers, etc.) and their availability at different instances of time, when the patient is being administered with multiple care needs.

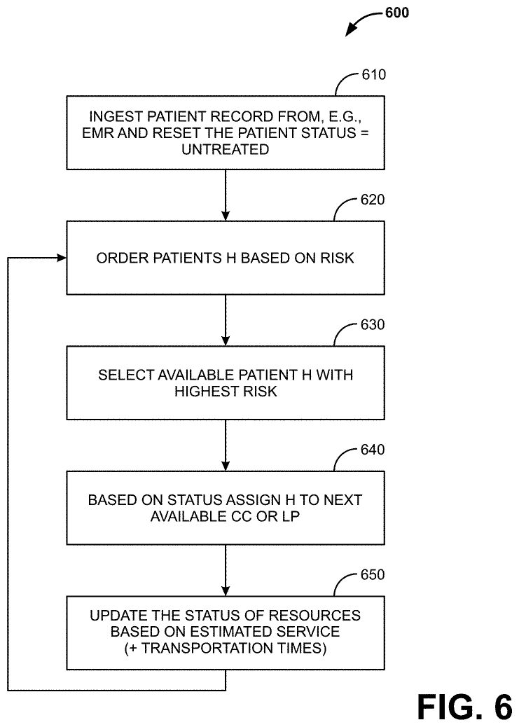

FIG. 6 is a flow diagram illustrating a process 600 to schedule human resources at a healthcare entity, according to an exemplary embodiment. FIG. 6 is described in conjunction with FIG. 2A, and FIG. 2B. In an embodiment, the scheduling the human resources may be for managing a patient treatment at a hospital. In an embodiment, patients may be admitted in the hospital based on electronic medical records (EMR). Upon admission, each patient's status may be reset as “untreated” or “under treatment.” (e.g., step 610 Ingest patient record from e.g., EMR and reset the patient status=untreated). For example, a patient may return from the care service and be admitted back to the hospital with updated attributes reflecting progression of disease or withdrawal of a malady. Upon admission, a patient admission monitoring system working in cooperation with the integrated optimization system of FIG. 2A may record the status change.

At step 620, the patients may be ordered based on risk (e.g., step 620 order patients H based on risk). For instance, the patients may be ranked based on the risk assessment. The integrated optimization system as described in FIG. 2A and FIG. 2B may execute operations to re-order the patient within a queue of patients who are waiting for treatment. As described with reference to FIG. 2B, the risk assessment may be computed based on the risk patient models 216. At step 630, patients with highest risk are selected. For example, the rank within the queue of the patients may be based on a customized key performance indicators (KPIs) related to the risk of the patient proceeding to an undesirable event. In an embodiment, one exemplary KPI metric for such ranking may be severity of the patient illness. The computation of risk assessment and the selection of the patient with the highest risk may be executed by the risk patient models 216. The queue is ordered such that patients with the highest risk are at the top of the queue, i.e., patients at higher risk are likely to be processed before the patients with a lower risk. In an embodiment, when the risk assessment level is lower, the integrated optimization system may rank the patients and reorder assignment to the care coordinators or the licensed providers. For instance, the integrated optimization system may reorder the patients with lower risk by either appropriately reserving the capacity for availing various services or by reserving slots for attending low-risk patients.

At step 640, based on an availability of the care coordinator or the licensed medical provider, the patient with the highest risk is assigned for the treatment. In an embodiment, the patient outcome optimization engine 206 may execute operations to perform the task of assigning the patient to the available care coordinator (CC) or the medical licensed provider (LP). At step 650, availability of resources is updated based on an estimation. For example, such estimation may be based on parameters such as the type of treatment, the time taken for the treatment, etc. (e.g., at step 650 update the status of resources based on estimated service (+transportation times)). For example, based on the type of risk assessment and the kind of service or the treatment the patient is waiting for, the patient status may be changed, and the patient status will appropriately reflected when the appropriate resource is available. In an embodiment, the patient may be assigned to the first slot available of the needed resource (e.g., care coordinators, medical licensed providers, etc.) and the status of the patient and the needed resource may appropriately be modified. In an embodiment, when the needed resource is not available, the patient continues to wait in the monitoring queue. The time of allocation of the resource to the patient may also be influenced by the socio-economic factors or budget constraints.

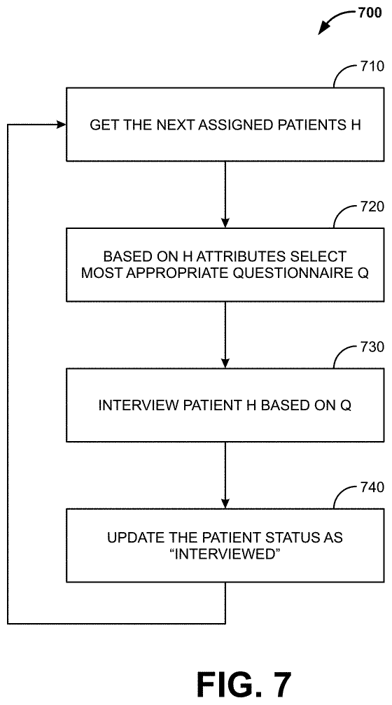

FIG. 7 is a flow diagram illustrating a process for managing a patient treatment by a care coordinator service, according to an exemplary embodiment. FIG. 7 is described in conjunction with FIG. 1, FIG. 2A, and FIG. 2B. In an embodiment, at step 710, the next assigned patient H may be attended. (e.g., at step 710 get the next assigned patients H). For example, the staff or the medical licensed professional associated with the care coordinator service may attend to the assigned patient based on the next available slot. At step 720, based on attributes of the patient H, the care coordinator may select an appropriate questionnaire Q from a set of questionnaires and may administer the questionnaire to the patient. At step 730, the patient H may be interviewed based on questionnaire Q. For example, the administration may use any medium appropriate for the dissemination based on patient attributes, time, and the constraints (e.g., cost or budget constraints). The administration may involve explicit patient participation, or the questionnaire could be filled by an automatic process inferring the answers from the latest data available for the patients including any sensory inputs (e.g., camera, microphone). The overall process of administration of questionnaires will be governed by taking appropriate consent from the patient and adhering to the appropriate privacy policies. At step 740, the status of the patient may be set as “interviewed.” For instance, upon conclusion of the “interview” process the patient attributes and status are updated and the corresponding patient is “returned” to the monitoring queue.

FIG. 8 is a flow diagram illustrating a process 800 for treating a patient by a medical licensed provider, according to an exemplary embodiment. FIG. 8 is described in conjunction with FIG. 1, FIG. 2A and FIG. 2B. In an embodiment, at step 810, the medical licensed provider may accept the assigned patient based on the availability in the next slot (e.g., at step 810 get the next assigned patients H). At step 820, the medical licensed provider selects an appropriate intervention (e.g., at step 820 based on H attributes select intervention). For example, based on the attributes of the patient and optional additional examinations, for example a physical examination, the appropriate intervention may be selected. At step 830, the status associated with the patient may be updated as “treated” (e.g., update the patient status as “treated”). Upon conclusion of the treatment process the patient attributes and status are updated and process 800 may loop back to the monitoring queue (e.g., step 810).

FIG. 9 is a block diagram illustrating a system 900 for generating a questionnaire for diagnosing a patient, according to an exemplary embodiment. FIG. 9 is described in conjunction with FIG. 1, FIG. 2A, FIG. 2B and FIG. 7. In an embodiment, the system 900 for generating a questionnaire may include a communicatively coupled arrangement of domain knowledge pack 920, a question bank 930 of questionnaires, an interview analysis engine 940, patient 950 under treatment, data or information related to a patient history 960. The care coordinator 990 may administer the patient 950 with the questions that may be generated based on situations, for example, dynamic situation based questions 970, and by an artificial intelligence recommended question 980. The patient answer 992 may be recorded by the care coordinator 990 administering the patient 950 with the questions. As described with reference to FIG. 7, the process 700 for managing the patient treatment may involve administering the patient with the questionnaire to enable care coordination interview. In an embodiment, an established guidelines associated with the risk factors for high-risk morbidities may be organized in a corresponding morbidity knowledge packs in a tabular format for each morbidity or morbidity combination of interest. A first column of the table may include the risk factor in a standard format. Additional columns of the table may include the numeric data such as to quantify the risk and extent of exacerbation.

In an embodiment, when the above described quantitative data does not exist, the additional column data may include qualitative information such as “low,” “medium,” “high” associated with the risk factors and the extent of exacerbation of the malady due to presence of the risk factor. Each morbidity knowledge pack (e.g., 920A, 920B, 920C) may further be organized into the domain knowledge pack 920. The domain knowledge pack 920 may enable creating the question bank 930. Each question in the question bank 930 may be associated with one or more risk factors associated with the specific morbidity knowledge pack (e.g., 920A, 920B, 920C). In an embodiment, the questions may be designed to elicit an answer (e.g., patient answer 992) that relates to the risk factor based on other factors, such as patient behavior, observations, knowledge, and information or data related to patient history 960. The questions may be formulated or generated such that the responses may include multiple options as choices. Each choice associated with each option may provision determining whether a risk factor is applicable to the patient. For example, when a sedentary lifestyle leads to an aggravation of diabetes, an illustrative question may include “Do you watch TV more than 5 hours of TV at home sitting in your couch?”.

In an embodiment, an interview analysis engine 940 may be implemented as the machine learning engine and may be trained with data that may include a combination of different lifestyle practices, scenarios, morbidities, patient history 960, etc. The interview analysis engine 940 may execute operations to select a question from the question bank 930 based on historical information associated with the patient history 960 of the patient 950 of interest and the overall context of the treatment of the patient 950. Each recommended question (e.g., 970 and 980) and its response (e.g., 992) may be organized and stored in a file in the data store. In an embodiment, the context of the overall interview process may include the current state of the response and stored in a file associated with the patient 950.

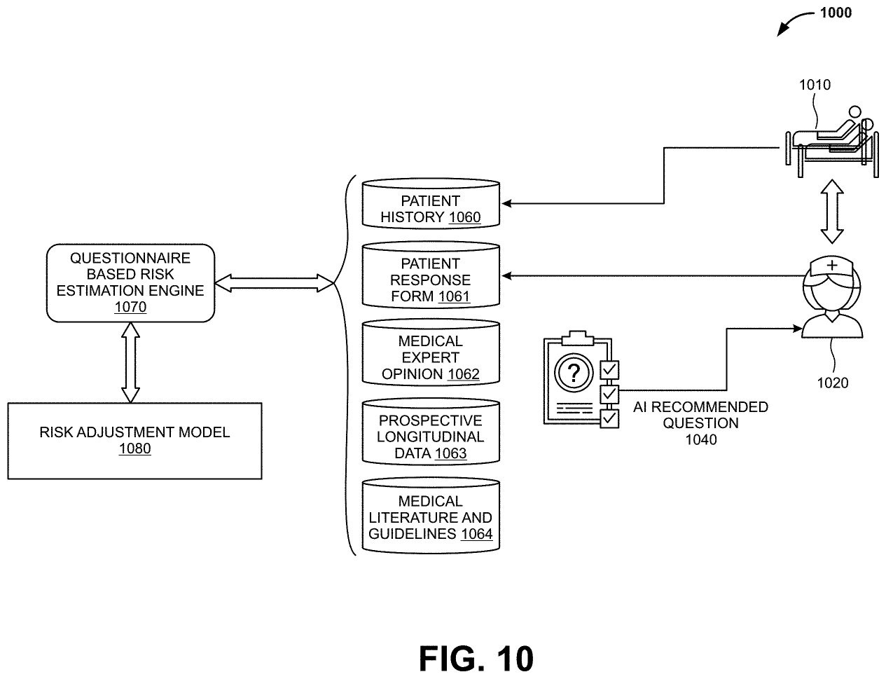

FIG. 10 is a block diagram showing a system 1000 for modifying a risk factor associated with a patient, according to an exemplary embodiment. FIG. 10 is described in conjunction with FIG. 1, FIG. 2A, FIG. 2B, FIG. 7 and FIG. 9. In an embodiment, the system 1000 includes a communicatively coupled arrangement of a patient 1010 under treatment, a care coordinator 1020, data or information including patient history 1060, patient response form 1061, medical expert opinion 1062, prospective longitudinal data 1063, medical literature and guidelines 1064, a risk estimation engine 1070, and a risk adjustment model 1080. As described with reference to FIG. 7 and FIG. 9, the patient 1010 that may be treated may be administered with the questionnaire (e.g., 1040) by the care coordinator 1020. In an embodiment, based on the response (e.g., patients answer 1030) to the questionnaire by the patient 1010, the risk estimation engine 1070 in cooperation with the risk adjustment model 1080 may execute operations to adjust or modify the risk factors associated with the patient 1010. For example, based on multidimensional data inputs (e.g., 1060, 1061, 1062, 1063, and 1064), administered questions (e.g., 1040) and the patient responses (e.g., 1030), the clinical outcomes of the patients from a prospective longitudinal study (e.g., 1063) may be used to dynamically model and train the risk adjustment model 1080.

In an embodiment, additional external sources of information such as facts from subject matter experts or medical providers (e.g., 1062), latest research and publication information (e.g., 1064) may be augmented to continually train the risk adjustment model 1080. The risk estimation engine 1070 may be configured to work in cooperation with the risk adjustment model 1080. The risk adjustment model 1080 may implement statistical regression analysis based on the responses (e.g., 1030) by the patient 1010. For example, when a majority percentage, for example, 80% of the patient population diagnosed with diabetes responded that they lead sedentary life may subsequently have further complications associated with diabetes. In such a scenario, a corresponding entry in the risk adjustment model 1080 may indicate that risk may be adjusted with an increased risk factor based on the fraction of concurring observations (for e.g., 80% of the patient population). In other embodiments, other known statistical techniques augmented with machine learning engines and risk adjustment model 1080 may be implemented to estimate the risk adjustment factor. In an embodiment, the risk adjustment model 1080 may enable modifying the questionnaire and the associated patient response (e.g., 1030). Based on the patient response (e.g., 1030), the current risk factors associated with the patient may be modified or revised.

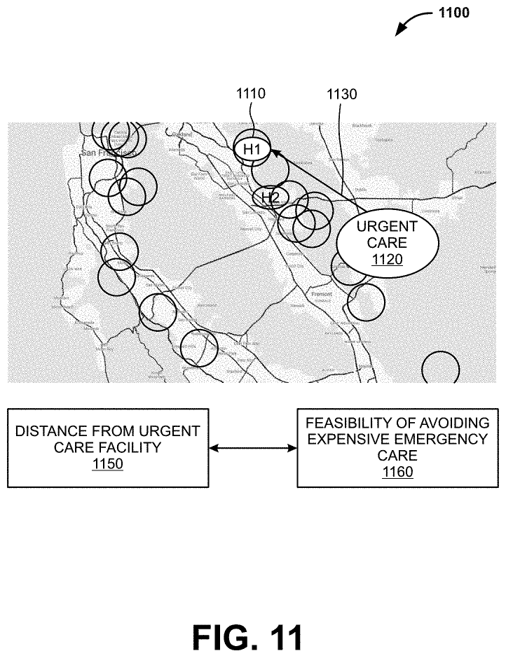

FIG. 11 is an illustration of an implementation of system 200A, 200B at a healthcare facility 1120 in a specific demographic region, according to an exemplary embodiment. FIG. 11 is described in conjunction with FIG. 2A, FIG. 2B, FIG. 8, FIG. 9, and FIG. 10. In an embodiment, the healthcare facility is shown as urgent care 1120. The patients H1 1110 and H2 1130 may be staying in the demographic region of the healthcare facility 1120. The system (e.g., 200A, 200B) implemented at the healthcare facility 1120 may store information related to the patients H1 1110 and H2 1130. As described with reference to FIG. 2A and FIG. 2B, the information or data may include historical information with information or data on risk factors associated with H1 1110 and H2 1130. The information associated with the patients may also include patient address information.

In an embodiment, the engines and the models, as described with reference to FIG. 2A and FIG. 2B, may execute operations to determine patients that may be at high risk that may need immediate attention. The engines and the models as described in FIG. 2A and FIG. 2B, may implement a mechanism to determine distance from the urgent care facility (e.g., 1150) and determine feasibility of avoiding expensive emergency care (e.g., 1160). The optimization engines 206 (e.g., 206, 206A, 206B, 206C) in cooperation with the other engines and the models may execute operations to optimize the outcomes. For example, in this scenario, optimizing outcome may correspond to determining feasibility of avoiding expensive emergency care 1160. In an embodiment, the urgent care facility or the healthcare facility (e.g., 1120) implementing the system (e.g., 100, 200A, 200B) may enable to proactively communicate with the patients H1 1110 and H2 1130 to keep track on the health conditions. Based on the scenario analysis 210 and the actionable insights based on the execution of operations by the system (e.g., 100, 200A, 200B) as described with reference to FIG. 2A and FIG. 2B, the healthcare facility 1120 may be able to reduce or mitigate the risks or proactively be able to prevent a situation leading to expensive healthcare related expenditures. In an embodiment, by reducing the associated risks through timely communication, the system (e.g., 100, 200A, 200B) may be able to optimize the outcomes for the healthcare facility (e.g., 1120).

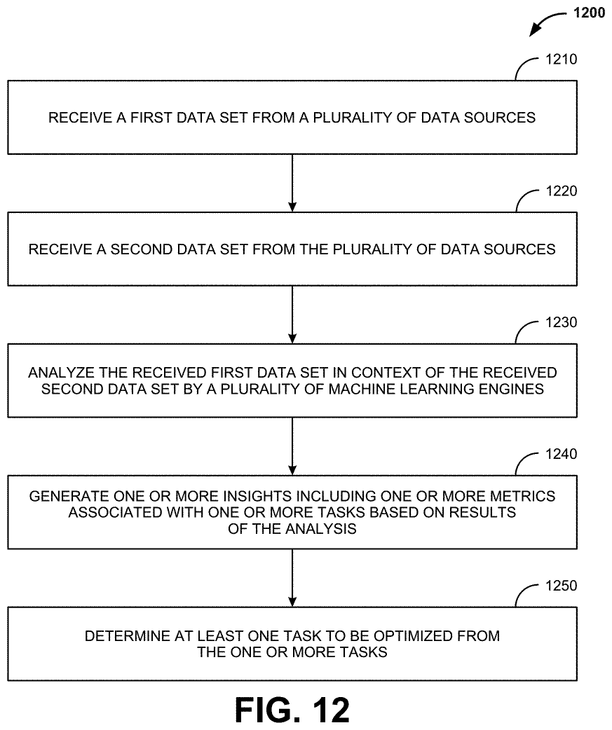

FIG. 12 is a flow diagram illustrating a process 1200 for optimizing outcomes for healthcare entities, according to an exemplary embodiment. FIG. 12 is described in conjunction with FIG. 1, FIG. 2A, FIG. 2B, FIG. 8 and FIG. 9. In an embodiment, at step 1210, a first data set from multiple data sources is received. The first data set may be associated, for example, with multiple medical records associated with multiple patients. At step 1220, a second data set from multiple data sources is received. The second data set may be associated with, for example, human resources, an associated attributes and/or associated constraints. At step 1230, the received first data set in context of the received second data is analyzed set by multiple machine learning engines. At step 1240, one or more insights including one or more metrics associated with one or more tasks based on results of the analysis are generated. The one or more tasks may be associated with one or more processes in the healthcare entity. At step 1250, at least one task to be optimized from the one or more tasks is determined. The optimization may improve the one or more metrics associated with the at least one task based on the generated one or more insights. The operational efficacies of the steps 1210, 1220, 1230, 1240, and 1250 of the process 1200 are as described in the detailed description of FIG. 1, FIG. 2A, FIG. 2B, FIG. 8 and FIG. 9.

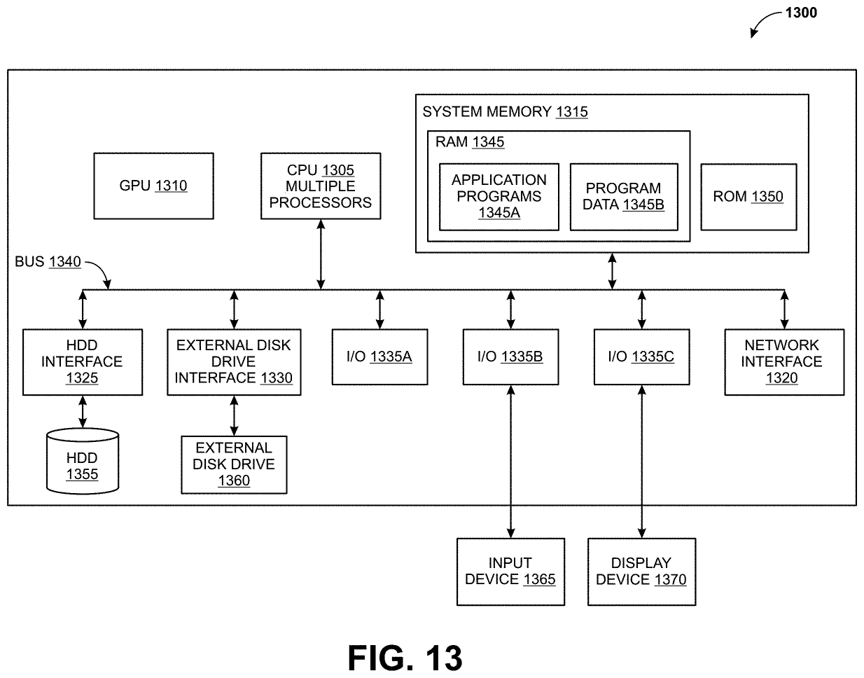

FIG. 13 shows an exemplary hardware configuration of computer 1300 that may be used to implement components of the system (100, 200A, 200B) for optimizing outcomes for healthcare entities, in accordance with exemplary embodiments. The computer 1300 shown in FIG. 13 includes CPU 1305, GPU 1310, system memory 1315, network interface 1320, hard disk drive (HDD) interface 1325, external disk drive interface 1330 and input/output (I/O) interfaces 1335A, 1335B, 1335C. These elements of the computer are coupled to each other via system bus 1340. The CPU 1305 may perform arithmetic, logic and/or control operations by accessing system memory 1315. The CPU 1305 may implement the processors of the exemplary devices and/or system described above. The GPU 1310 may perform operations for processing graphic or AI tasks. In case computer 1300 is used for implementing exemplary central processing device, GPU 1310 may be GPU 1310 of the exemplary central processing device as described above. The computer 1300 does not necessarily include GPU 1310, for example, in case computer 1300 is used for implementing a device other than central processing device. The system memory 1315 may store information and/or instructions for use in combination with the CPU 1305. The system memory 1315 may include volatile and non-volatile memory, such as random-access memory (RAM) 1345 and read only memory (ROM) 1350. A basic input/output system (BIOS) containing the basic routines that help to transfer information between elements within the computer 1300, such as during start-up, may be stored in ROM 1350. The system bus 1340 may be any of several types of bus structures including a memory bus or memory controller, a peripheral bus, and a local bus using any of a variety of bus architectures.

The computer may include network interface 1320 for communicating with other computers and/or devices via a network.

Further, the computer may include hard disk drive (HDD) 1355 for reading from and writing to a hard disk (not shown), and external disk drive 1360 for reading from or writing to a removable disk (not shown). The removable disk may be a magnetic disk for a magnetic disk drive or an optical disk such as a CD ROM for an optical disk drive. The HDD 1355 and external disk drive 1360 are connected to the system bus 1340 by HDD interface 1325 and external disk drive interface 1330, respectively. The drives and their associated non-transitory computer-readable media provide non-volatile storage of computer-readable instructions, data structures, program modules and other data for the general-purpose computer. The relevant data may be organized in a database, for example a relational or object database.

Although the exemplary environment described herein employs a hard disk (not shown) and an external disk (not shown), it should be appreciated by those skilled in the art that other types of computer readable media which can store data that is accessible by a computer, such as magnetic cassettes, flash memory cards, digital video disks, random access memories, read only memories, and the like, may also be used in the exemplary operating environment.

Several program modules may be stored on the hard disk, external disk, ROM 1350, or RAM 1345, including an operating system (not shown), one or more application programs 1345A, other program modules (not shown), and program data 1345B. The application programs may include at least a part of the functionality as described above.

The computer 1300 may be connected to input device 1365 such as mouse and/or keyboard and display device 1370 such as liquid crystal display, via corresponding I/O interfaces 1335A to 1335C and the system bus 1340. In addition to an implementation using a computer 1300 as shown in FIG. 13, a part or all the functionality of the exemplary embodiments described herein may be implemented as one or more hardware circuits. Examples of such hardware circuits may include but are not limited to: Large Scale Integration (LSI), Reduced Instruction Set Circuits (RISC), Application Specific Integrated Circuit (ASIC) and Field Programmable Gate Array (FPGA).

One or more embodiments are now described with reference to the drawings, wherein like reference numerals are used to refer to like elements throughout. In the following description, for purposes of explanation, numerous specific details are set forth to provide a thorough understanding of the various embodiments. It is evident, however, that the various embodiments can be practiced without these specific details (and without applying to any networked environment or standard).

As used in this application, in some embodiments, the terms “component,” “system” and the like are intended to refer to, or comprise, a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, computer-executable instructions, a program, and/or a computer. By way of illustration and not limitation, both an application running on a server and the server can be a component.

The above descriptions and illustrations of embodiments, including what is described in the Abstract, is not intended to be exhaustive or to limit the one or more embodiments to the precise forms disclosed. While specific embodiments of, and examples for, the one or more embodiments are described herein for illustrative purposes, various equivalent modifications are possible within the scope, as those skilled in the relevant art will recognize. These modifications can be made considering the above detailed description. Rather, the scope is to be determined by the following claims, which are to be interpreted in accordance with established doctrines of claim construction.

What is claimed is:

1. A system, comprising:
a processor;
a memory storing instructions that are executed by the processor, to:
receive a first data set from a plurality of data sources, wherein the first data set is associated with a plurality of medical records associated with a plurality of patients;
receive a second data set from the plurality of data sources, wherein the second data set is associated with a plurality of resources, an associated plurality of attributes and an associated plurality of constraints;
analyze the received first data set in context of the received second data set by a plurality of machine learning engines;
generate one or more insights including one or more metrics associated with one or more tasks based on results of the analysis, wherein the one or more tasks are associated with one or more processes in a healthcare entity; and
based on the generated one or more insights, determine at least one task to be optimized from the one or more tasks, wherein the optimization improves the one or more metrics associated with the at least one task;
wherein the plurality of machine learning engines are trained based on training data related to the first data set and the second data set, and the plurality of machine learning engines are re-trained continually based on an updated or modified first data set and an updated or modified second data set.

2. The system of claim 1, further comprises based on the analysis of the first data set in context of the second data set, generate a questionnaire to determine a treatment plan for the plurality of patients.

3. The system of claim 1, further comprises based on the results of the analysis of the first data set in context of the second data set, prioritize treating at least one patient from the plurality of patients, wherein the prioritization of the treatment is based on a plurality of risk factors associated with the at least one patient.

4. The system of claim 1, further comprises modify at least one risk factor from the plurality of risk factors associated with the at least one patient based on the results of the analysis of the first data set in context of the second data set.

5. The system of claim 1, further comprises based on the results of the analysis of the first data set, generate a risk estimation associated with at least one patient from the plurality of patients.

6. The system of claim 1, further comprises based on the results of the analysis of the first data set in context of the second data set, generate a schedule for treating the at least one patient.

7. The system of claim 1, wherein the analyzing step is performed by the plurality of machine learning engines including one or more of an ACO quality management analysis engine, a population disease risk management engine, population disease cost management engine, a healthcare quality optimization engine, a cost optimization engine, and a gap analysis engine.

8. A method, comprising:
receiving a first data set from a plurality of data sources, wherein the first data set is associated with a plurality of medical records associated with a plurality of patients;
receiving a second data set from the plurality of data sources, wherein the second data set is associated with a plurality of resources, an associated plurality of attributes and an associated plurality of constraints;
analyzing the received first data set in context of the received second data set by a plurality of machine learning engines;
generating one or more insights including one or more metrics associated with one or more tasks based on results of the analysis, wherein the one or more tasks are associated with one or more processes in a healthcare entity; and
based on the generated one or more insights, determining at least one task to be optimized from the one or more tasks, wherein the optimization improves the one or more metrics associated with the at least one task;
wherein the plurality of machine learning engines are trained based on training data related to the first data set and the second data set, and the plurality of machine learning engines are re-trained continually based on an updated or modified first data set and an updated or modified second data set.

9. The method of claim 8, further comprising, based on the analysis of the first data set in context of the second data set, generating a questionnaire to determine a treatment plan for the plurality of patients.

10. The method of claim 8, further comprising based on the results of the analysis of the first data set in context of the second data set, prioritizing treating at least one patient from the plurality of patients, wherein the prioritization of the treatment is based on a plurality of risk factors associated with the at least one patient.

11. The method of claim 8, further comprising modifying at least one risk factor from the plurality of risk factors associated with the at least one patient based on the results of the analysis of the first data set in context of the second data set.

12. The method of claim 8, further comprising based on the results of the analysis of the first data set, generating a risk estimation associated with at least one patient from the plurality of patients.

13. The method of claim 8, further comprising based on the results of the analysis of the first data set in context of the second data set, generating a schedule for treating the at least one patient.

14. The method of claim 8, wherein the analyzing step is performed by the plurality of machine learning engines including one or more of an ACO quality management analysis engine, a population disease risk management engine, population disease cost management engine, a healthcare quality optimization engine, a cost optimization engine, and a gap analysis engine.

15. A non-transitory computer-readable device having instructions stored thereon that, when executed by at least one computing device, cause the at least one computing device to perform operations comprising:
receiving a first data set from a plurality of data sources, wherein the first data set is associated with a plurality of medical records associated with a plurality of patients;
receiving a second data set from the plurality of data sources, wherein the second data set is associated with a plurality of resources, an associated plurality of attributes and an associated plurality of constraints;
analyzing the received first data set in context of the received second data set by a plurality of machine learning engines;
generating one or more insights including one or more metrics associated with one or more tasks based on results of the analysis, wherein the one or more tasks are associated with one or more processes in a healthcare entity; and
based on the generated one or more insights, determining at least one task to be optimized from the one or more tasks, wherein the optimization improves the one or more metrics associated with the at least one task;
wherein the plurality of machine learning engines are trained based on training data related to the first data set and the second data set, and the plurality of machine learning engines are re-trained continually based on an updated or modified first data set and an updated or modified second data set.

16. The non-transitory computer-readable device of claim 15, further comprising: based on the analysis of the first data set in context of the second data set, generating a questionnaire to determine a treatment plan for the plurality of patients.

17. The non-transitory computer-readable device of claim 15, further comprising, based on the results of the analysis of the first data set in context of the second data set, prioritizing treating at least one patient from the plurality of patients, wherein prioritization of the treatment is based on a plurality of risk factors associated with the at least one patient.

18. The non-transitory computer-readable device of claim 15, further comprising, modifying at least one risk factor from the plurality of risk factors associated with the at least one patient based on the results of the analysis of the first data set in context of the second data set.

19. The non-transitory computer-readable device of claim 15, further comprising, based on the results of the analysis of the first data set, generating a risk estimation associated with at least one patient from the plurality of patients.

20. The non-transitory computer-readable device of claim 15, further comprising, based on the results of the analysis of the first data set in context of the second data set, generating a schedule for treating the at least one patient.
